SEQUENCE LISTING (3 sequences)

SEQ ID NO: 1 - protein, 836 aa, Homo sapiens
MTTLAGAVPRMMRPGPGQNYPRSGFPLEVSTPLGQGRVNQLGGVFINGRPLPNHIRHKIVEMAHHGIRPCVISRQLRVSHGCVSKILCRYQETGSIRPGAIGGSKPKQVTTPDVEKKIEEYKRENPGMFSWEIRDKLLKDAVCDRNTVPSVSSISRILRSKFGKGEEEEADLERKEAEESEKKAKHSIDGILSERASAPQSDEGSDIDSEPDLPLKRKQRRSRTTFTAEQLEELERAFERTHYPDIYTREELAQRAKLTEARVQVWFSNRRARWRKQAGANQLMAFNHLIPGGFPPTAMPTLPTYQLSETSYQPTSIPQAVSDPSSTVHRPQPLPPSTVHQSTIPSNPDSSSAYCLPSTRHGFSSYTDSFVPPSGPSNPMNPTIGNGLSPQNSIRHNLSLHSKFIRVQNEGTGKSSWWMLNPEGGKSGKSPRRRAASMDNNSKFAKSRSRAAKKKASLQSGQEGAGDSPGSQFSKWPASPGSHSNDDFDNWSTFRPRTSSNASTISGRLSPIMTEQDDLGEGDVHSMVYPPSAAKMASTLPSLSEISNPENMENLLDNLNLLSSPTSLTVSTQSSPGTMMQQTPCYSFAPPNTSLNSPSPNYQKYTYGQSSMSPLPQMPIQTLQDNKSSYGGMSQYNCAPGLLKELLTSDSPPHNDIMTPVDPGVAQPNSRVLGQNVMMGPNSVMSTYGSQASHNKMMNPSSHTHPGHAQQTSAVNGRPLPHTVSTMPHTSGMNRLTQVKTPVQVPLPHPMQMSALGGYSSVSSCNGYGRMGLLHQEKLPSDLDGMFIERLDCDMESIIRNDLMDGDTLDFNFDNVLPNQSFPHSVKTTTHSWVSG

SEQ ID NO: 2 - protein, 271 aa, Homo sapiens
XEEEKIFGRGSRQRRDVDYSDALTEKQWLRAIEDGNLEEMEEEVRLKKRKRRRNVDKDPAKEDVEKAKKRRGRPPAEKLSPNPPKLTKQMNAIIDTVINYKDSSGRQLSEVFIQLPSRKELPEYYELIRKPVDFKKIKERIRNHKYRSLGDLEKDVMLLCHNAQTFNLEGSQIYEDSIVLQSVFKSARQKIAKEEESEDESNEEEEEEDEEESESEAKSVKVKIKLNKKDDKGRDKGKGKKRPNRGKAKPVVSDFDSDEEQDERVSVADWD

SEQ ID NO: 3 - protein, 157 aa, Homo sapiens
MSTPDPPLGGTPRPGPSPGPGPSPGAMLGPSPGPSPGSAHSMMGPSPGPPSAGHPIPTQGPGGYPQDNMHQMHKPMESMHEKGMSDDPRYNQMKGMGMRSGGHAGMGPPPSPMDQHSQGYPSPLGGSEHASSPVPASGPSSGPQMSSGPGGAPLDGA

(19) United States
(12) Patent Application Publication
(10) Pub. No.: US 2026/0207753 A1
(43) Pub. Date: Jul. 23, 2026

(54) PROTAC DEGRADATION OF FACTORS PAX3 AND FOXO1

(71) Applicants: THE RESEARCH INSTITUTE AT NATIONWIDE CHILDREN'S HOSPITAL, Columbus, OH (US); DANA-FARBER CANCER INSTITUTE, INC., BOSTON, MA (US)

(72) Inventors: Benjamin STANTON, COLUMBUS, OH (US); Benjamin Douglas SUNKEL, COLUMBUS, OH (US); Jun QI, Boston, MA (US)

(21) Appl. No.: 19/138,815

(22) PCT Filed: Dec. 14, 2023
(86) PCT No.: PCT/US2023/084120

§ 371 Date: Jun. 13, 2025

Related U.S. Application Data
(60) Provisional application No. 63/432,490, filed on Dec. 14, 2022.

(51) Int. Cl. A61K 47/55; A61P 35/00; C12Q 1/6869; G01N 21/64; G01N 33/58

(52) U.S. Cl. CPC A61K 47/55; A61P 35/00; C12Q 1/6869; G01N 21/6458; G01N 33/582; C12Q 2600/136

(57) ABSTRACT

A method of treating rhabdomyosarcoma in a subject is described that includes administering a therapeutically effective amount of a PROTAC compound that results in the degradation of FOXO1 or a PAX3-FOXO1 fusion protein. A method of studying the chromatin-level effects of switching defective and sucrose nonfermenting (SWI/SNF) inactivators, comprising degrading ATPase using a PROTAC compound, and measuring the biological effect of the loss of SWI/SNF complexes is also described.

### CROSS-REFERENCE TO RELATED APPLICATIONS

This application claims priority to U.S. Provisional Application Ser. No. 63/432,490, filed on Dec. 14, 2022, which is hereby incorporated by reference in its entirety.

### SEQUENCE LISTING

The instant application contains a Sequence Listing which has been submitted in ASCII format via EFS-Web and is hereby incorporated by reference in its entirety.

### BACKGROUND

Rhabdomyosarcoma (RMS) is a devastating pediatric cancer with the most aggressive form of the disease being genetically defined by fusions between PSX3/7 and FOXO1. This rare pediatric tumor has a poor prognosis, with survival rates at 30-50%, that have not improved in several decades. In fusion-positive RMS (FP-RMS), the early targeting function of the primary fusion protein PAX3-FOXO1 has remained unclear. Accordingly, there has been a critical need to precisely define the requirements for PAX3-FOXO1 function at the chromatin level. PAX3-FOXO1 uses super enhancers to set up autoregulatory loops in collaboration with the master transcription factors MYOG, MYOD, and MYCN. Gryder et al., Cancer Discov. 7, 884-899 (2017). However, the immediate targeting mechanisms of PAX3-FOXO1 in the context of chromatin accessibility have yet to be assessed in a temporally-controlled system.

Targeted protein degradation is attracting substantial interest due to its potential to therapeutically modulate proteins that have proven difficult to target. A major class of molecules that may enable such proteins to be modulated through protein degradation are known as proteolysis-targeting chimeras (PROTAC)s. The first synthetic protein, dubbed Protac-1, was developed in 2001. Sakamoto et al., Proc. Natl. Acad. Sci. USA 98, 8554-8559 (2001). Since then, a wide variety of PROTAC molecules have been developed. Bekes et al., Nat Rev Drug Discov., 21(3):181-200 (2022).

Therapy for the aggressive alveolar RMS subtype relies upon surgery, radiation, and broadly toxic drugs. Arndt et al., J Clin Oncol, 27(31):5182-8 (2009). However, the 5-year survival rate for subjects receiving treatment is typically only about 50%. Accordingly, there remains a need for compounds useful for treating RMS.

### SUMMARY OF THE INVENTION

The inventors have demonstrated that PROTAC compounds known to target the ATPases SMARCA4 and SMARCA2 can also function as ATPase-degrading PROTACs. An incidental early finding is that these PROTACs also result in the loss of the rhabdomyosarcoma-driving transcription factors including PAX3-FOXO1 and MYOD1. Thus, inventors' work indicates that SMARCA4-31 and DL-dS2-4 have two concurrent activities.

The first of these activities is SMARCA4 and SMARCA2 depletion. This activity underlies the claim that these PROTACs could be utilized for understanding ATP-dependent remodeling in rhabdomyosarcoma. The second of these activities is depletion of the BAF associated transcription factors (e.g., PAX3-FOXO1 and MYOD1). This unexpected activity enables the use of these PROTACs for the purposes of understanding the activity of BAF associated transcription factors in rhabdomyosarcoma, and treating rhabdomyosarcoma.

The inventors have targeted core catalytic subunits of ATP-dependent chromatin remodeling complexes in each major subtype of rhabdomyosarcoma with the PROTAC degraders SMARCA4-31 and DL-dS2-4 to enable concomitant degradation of (1) the driver oncogene in the alveolar subtype, PAX3-FOXO1, and (2) the FOXO1 transcription factor in the embryonal subtype. These effects are accompanied by defects in rhabdomyosarcoma cell proliferation. The degradation of PAX3-FOXO1 is only partially rescued with removal of the PROTACS. FOXO1 or PAX3-FOXO1 fusions have not previously been targeted for rapid degradation. This provides both a new process of BAF-proximity degradation, and a new process of PAX3-FOXO1 degradation.

### BRIEF DESCRIPTION OF THE FIGURES

The present invention may be more readily understood by reference to the following figures, wherein:

FIG. 1 provides a schematic representation describing the modular constituents of each PROTAC. In both cases, a thalidomide moiety is used to provide CRBN-targeting activity. For SMARCA4-31, SMARCA4/SMARCA2 bromodomain-targeting is achieved by linking thalidomide to PFI-3. In the case of DL-dS2-4, SMARCA4/SMARCA2 ATPase domain-targeting is achieved by linking thalidomide to BRM014.

FIG. 2 provides an image showing that the PROTAC compounds tested showed significant degradation of BRD9, SMARCA2, and SMARCA4 in RH4 and RD cells.

FIG. 3 provides an image showing that the PROTAC compounds tested showed significant degradation of BRD9, SMARCA2, and SMARCA4 in RH30 and CTR cells.

FIG. 4 provides an image showing the on-target activity of the PROTAC compounds in RH4 cells over 24 hours

FIG. 5 provides a set of graphs showing that the reversible disruption of the SWU/SNF complex results in altered chromatin accessibility.

FIGS. 6A and 6B provide images showing full PAX3-FOXO1 loss at lower PROTAC doses and over shorter treatment periods.

FIGS. 7A and 7B provide graphs demonstrating sub-micromolar efficacy of SMARCA4-31 and DL-dS2-4 in inhibiting growth of two rhabdomyosarcoma cell lines, RH4 and SMS-CTR.

### DETAILED DESCRIPTION OF THE INVENTION

The present invention provides a method of treating rhabdomyosarcoma in a subject that includes administering a therapeutically effective amount of a PROTAC compound that results in the degradation of FOXO1 or a PAX3-FOXO1 fusion protein. The present invention also provides a method of studying the chromatin-level effects of switching defective and sucrose nonfermenting (SWI/SNF) inactivators, comprising degrading ATPase using a PROTAC compound, and measuring the effect of the loss of SWI/SNF complexes.

Unless otherwise defined, all technical and scientific terms used herein have the same meaning as commonly understood by one of ordinary skill in the art to which these exemplary embodiments belong. The terminology used in the description herein is for describing particular exemplary embodiments only and is not intended to be limiting of the exemplary embodiments. As used in the specification and the appended claims, the singular forms “a,” “an,” and “the” are intended to include the plural forms as well, unless the context clearly indicates otherwise.

Throughout this application, the term “about” is used to indicate that a value includes the standard deviation of error for the device or method being employed to determine the value, except that the value will never deviate by more than 5% from the value cited.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

“Treating”, as used herein, means ameliorating the effects of, or delaying, halting or reversing the progress of a disease or disorder. Treatment includes prophylactic treatment of subjects diagnosed with cancer who have not yet exhibited symptoms of the disease, and non-prophylactic treatment of subjects who have exhibited symptoms. The word encompasses reducing the severity of a symptom of a disease or disorder and/or the frequency of a symptom of a disease or disorder. A subject is successfully “treated” for a disease or disorder if the subject shows observable and/or measurable reduction in or absence of one or more signs and symptoms of a particular disease or condition.

The terms “therapeutically effective” and “pharmacologically effective” are intended to qualify the amount of each agent which will achieve the goal of decreasing disease severity while avoiding adverse side effects such as those typically associated with alternative therapies. The therapeutically effective amount may be administered in one or more doses. An effective amount, on the other hand, is an amount sufficient to provide a significant chemical effect.

A “subject”, as used therein, can be a human or non-human animal. Non-human animals include, for example, livestock and pets, such as ovine, bovine, porcine, canine, feline and murine mammals, as well as reptiles, birds and fish. Preferably, the subject is human. Subjects can also be selected from different age groups. For example, the subject can be a child, adult, or elderly subject.

The term “gene,” as used herein, means one or more sequence(s) of nucleotides in a genome that together encode one or more expressed molecule, e.g., an RNA, or polypeptide.

The gene can include coding sequences that are transcribed into RNA which may then be translated into a polypeptide sequence, and can include associated structural or regulatory sequences that aid in replication or expression of the gene.

“Nucleic acid” or “oligonucleotide” or “polynucleotide”, as used herein, may mean at least two nucleotides covalently linked together. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof. The term “nucleotide sequence,” as used herein, refers to an oligonucleotide, nucleotide, or polynucleotide of single-stranded or double stranded DNA or RNA, or fragments thereof.

As used herein, the term “alkylene” refers to a straight or branched divalent hydrocarbon chain linking the rest of the molecule to a radical group, consisting solely of carbon and hydrogen, containing no unsaturation and having from one to 12 carbon atoms, for example, methylene, ethylene, propylene, n-butylene, and the like. The alkylene chain may be attached to the rest of the molecule through a single bond and to the radical group through a single bond. To the extent not described otherwise for any one or more groups, in some embodiments, the alkylene group contains one to 8 carbon atoms (C₁-C₈ alkylene). In other embodiments, an alkylene group contains one to 5 carbon atoms (C₁-C₅ alkylene). In other embodiments, an alkylene group contains one to 4 carbon atoms (C₁-C₄ alkylene). In other embodiments, an alkylene contains one to three carbon atoms (C₁-C₃ alkylene). In other embodiments, an alkylene group contains one to two carbon atoms (C₁-C₂ alkylene). In other embodiments, an alkylene group contains one carbon atom (C₁ alkylene).

As used herein, the term “alkenyl” refers to a linear or branched-chain monovalent hydrocarbon radical with at least one carbon-carbon double bond. An alkenyl includes radicals having “cis” and “trans” orientations, or alternatively. “E” and “Z” orientations. To the extent not described otherwise for any one or more groups, in one example, the alkenyl radical is a C₂-C₁₈ group. In other embodiments, the alkenyl radical is a C₂-C₁₂, C₂-C₁₀, C₂-C₈, C₂-C₆ or C₂-C₃ group. Examples include ethenyl or vinyl, prop-1-enyl, prop-2-enyl, 2-methylprop-1-enyl, but-1-enyl, but-2-enyl, but-3-enyl, buta-1, 3-dienyl, 2-methylbuta-1,3-diene, hex-1-enyl, hex-2-enyl, hex-3-enyl, hex-4-enyl and hexa-1,3-dienyl.

The terms “alkoxyl” or “alkoxy” as used herein refer to an alkyl group, as defined above, having an oxygen radical attached thereto. Representative alkoxyl groups include methoxy, ethoxy, propyloxy, tert-butoxy and the like. An “ether” is two hydrocarbyl groups covalently linked by an oxygen. Accordingly, the substituent of an alkyl that renders that alkyl an ether is or resembles an alkoxyl, such as can be represented by one of —O-alkyl. —O-alkenyl, and —O-alkynyl.

As used herein, the term “alkoxylene” refers to a saturated monovalent aliphatic radicals of the general formula (—O—CₙH₂ₙ—) where n represents an integer (e.g., 1, 2, 3, 4, 5, 6, or 7) and is inclusive of both straight-chain and branched-chain radicals. The alkoxylene chain may be attached to the rest of the molecule through a single bond and to the radical group through a single bond. To the extent not described otherwise for any one or more groups, in some embodiments, the alkoxylene group contains one to 3 carbon atoms (—O—C₁-C₃ alkoxylene). In other embodiments, an alkoxylene group contains one to 5 carbon atoms (—O—C₁-C₅ alkoxylene).

As used herein, the term “cyclic group” broadly refers to any group that used alone or as part of a larger moiety, contains a saturated, partially saturated or aromatic ring system e.g., carbocyclic (cycloalkyl, cycloalkenyl), heterocyclic (heterocycloalkyl, heterocycloalkenyl), aryl and heteroaryl groups. Cyclic groups may have one or more (e.g., fused) ring systems. Thus, for example, to the extent not described otherwise for any one or more groups, a cyclic group can contain one or more carbocyclic, heterocyclic, aryl or heteroaryl groups.

As used herein, the term “carbocyclic” (also “carbocyclyl”) refers to a group that used alone or as part of a larger moiety, contains a saturated, partially unsaturated, or aromatic ring system having 3 to 20 carbon atoms, that is alone or part of a larger moiety (e.g., an alkcarbocyclic group). The term carbocyclyl includes mono-, bi-, tri-, fused, bridged, and spiro-ring systems, and combinations thereof. To the extent not described otherwise for any one or more groups, in one embodiment, carbocyclyl includes 3 to 15 carbon atoms (C₃-C₁₃). In one embodiment, carbocyclyl includes 3 to 12 carbon atoms (C₃-C₂). In another embodiment, carbocyclyl includes C₃-C₈, C₃-C₁₀ or C₅-C₁₀. In another embodiment, carbocyclyl, as a monocycle, includes C₃-C₈, C₃-C₆ or C₅-C₆. In some embodiments, carbocyclyl, as a bicycle, includes C₇-C₁₂. In another embodiment, carbocyclyl, as a spiro system, includes C₅-C₁₂.

Representative examples of monocyclic carbocyclyls include cyclopropyl, cyclobutyl, cyclopentyl, 1-cyclopent-1-enyl, 1-cyclopent-2-enyl, 1-cyclopent-3-enyl, cyclohexyl, perdeuteriocyclohexyl, 1-cyclohex-1-enyl, 1-cyclohex-2-enyl, 1-cyclohex-3-enyl, cyclohexadienyl, cycloheptyl, cyclooctyl, cyclononyl, cyclodecyl, cycloundecyl, phenyl, and cyclododecyl; bicyclic carbocyclyls having 7 to 12 ring atoms include [4,3], [4,4], [4,5], [5,5], [5,6] or [6,6] ring systems, such as for example bicyclo[2.2.1]heptane, bicyclo[2.2.2]octane, naphthalene, and bicyclo(3.2.2)nonane. Representative examples of spiro carbocyclyls include spiro[2.2]pentane, spiro[2.3]hexane, spiro[2.4]heptane, spiro[2.5]octane and spiro[4.5]decane. The term carbocyclyl includes aryl ring systems as defined herein. The term carbocycyl also includes cycloalkyl rings (e.g., saturated or partially unsaturated mono-, bi-, or spiro-carbocycles). The term carbocyclic group also includes a carbocyclic ring fused to one or more (e.g., 1, 2 or 3) different cyclic groups (e.g., aryl or heterocyclic rings), where the radical or point of attachment is on the carbocyclic ring.

Thus, the term carbocyclic also embraces carbocyclylalkyl groups which as used herein refer to a group of the formula —Re-carbocyclyl where Rc is an alkylene chain. The term carbocyclic also embraces carbocyclylalkoxy groups which as used herein refer to a group bonded through an oxygen atom of the formula —O— Rc-carbocyclyl where Rc is an alkylene chain.

As used herein, the term “aryl” used alone or as part of a larger moiety (e.g., “aralkyl”, wherein the terminal carbon atom on the alkyl group is the point of attachment, e.g., a benzyl group), “aralkoxy” wherein the oxygen atom is the point of attachment, or “aroxyalkyl” wherein the point of attachment is on the aryl group) refers to a group that includes monocyclic, bicyclic or tricyclic, carbon ring system, that includes fused rings, wherein at least one ring in the system is aromatic. In some embodiments, the aralkoxy group is a benzoxy group. The term “aryl” may be used interchangeably with the term “aryl ring”. To the extent not described otherwise for any one or more groups, in one embodiment, aryl includes groups having 6-18 carbon atoms. In another embodiment, aryl includes groups having 6-10 carbon atoms. Examples of aryl groups include phenyl, naphthyl, anthracyl, biphenyl, phenanthrenyl, naphthacenyl, 1,2,3,4-tetrahydronaphthalenyl, IH-indenyl, 2,3-dihydro-1H-indenyl, naphthyridinyl, and the like, which may be substituted or independently substituted by one or more substituents described herein. A particular aryl is phenyl. In some embodiments, an aryl group includes an aryl ring fused to one or more (e.g., 1, 2 or 3) different cyclic groups (e.g., carbocyclic rings or heterocyclic rings), where the radical or point of attachment is on the aryl ring. The structure of any aryl group that is capable of having double bonds positioned differently is considered so as to embrace any and all such resonance structures.

Thus, the term aryl embraces aralkyl groups (e.g., benzyl) which as disclosed above refer to a group of the formula —Rc-aryl where Rc is an alkylene chain such as methylene or ethylene. In some embodiments, the aralkyl group is an optionally substituted benzyl group.

The term aryl also embraces aralkoxy groups which as used herein refer to a group bonded through an oxygen atom of the formula —O—Rc-aryl where Rc is an alkylene chain such as methylene or ethylene.

As used herein, the term “heterocyclyl” refers to a “carbocyclyl” that used alone or as part of a larger moiety, contains a saturated, partially unsaturated or aromatic ring system, wherein one or more (e.g., 1, 2, 3, or 4) carbon atoms have been replaced with a heteroatom (e.g., O, N, N(O), S, S(O), or S(O)₂). The term heterocyclyl includes mono-, bi-, tri-, fused, bridged, and spiro-ring systems, and combinations thereof. To the extent not described otherwise for any one or more groups, in some embodiments, a heterocyclyl refers to a 3 to 15 membered heterocyclyl ring system. In some embodiments, a heterocyclyl refers to a 3 to 12 membered heterocyclyl ring system. In some embodiments, a heterocyclyl refers to a saturated ring system, such as a 3 to 12 membered saturated heterocyclyl ring system. In some embodiments, a heterocyclyl refers to a heteroaryl ring system, such as a 5 to 14 membered heteroaryl ring system. The term heterocyclyl also includes C₃-C₈ heterocycloalkyl, which is a saturated or partially unsaturated mono-, bi-, or spiro-ring system containing 3-8 carbons and one or more (1, 2, 3 or 4) heteroatoms.

To the extent not described otherwise for any one or more groups, in some embodiments, a heterocyclyl group includes 3-12 ring atoms and includes monocycles, bicycles, tricycles and spiro ring systems, wherein the ring atoms are carbon, and one to 5 ring atoms is a heteroatom such as nitrogen, sulfur or oxygen. Thus, the term heterocyclic embraces N-heterocyclyl groups which as used herein refer to a heterocyclyl group containing at least one nitrogen and where the point of attachment of the heterocyclyl group to the rest of the molecule is through a nitrogen atom in the heterocyclyl group. Representative examples of N-heterocyclyl groups include 1-morpholinyl, 1-piperidinyl, 1-piperazinyl, 1-pyrrolidinyl, pyrazolidinyl, imidazolinyl and imidazolidinyl.

As used herein, the term “heteroaryl” used alone or as part of a larger moiety (e.g., “heteroaryl alkyl” (also “heteroaralkyl”), or “heteroarylalkoxy” (also “heteroaralkoxy”), refers to a monocyclic, bicyclic or tricyclic ring system having 5 to 14 ring atoms, wherein at least one ring is aromatic and contains at least one heteroatom. To the extent not described otherwise for any one or more groups, in one embodiment, heteroaryl includes 5-6 membered monocyclic aromatic groups where one or more ring atoms is nitrogen, sulfur or oxygen. Representative examples of heteroaryl groups include thienyl, furyl, imidazolyl, pyrazolyl, thiazolyl, isothiazolyl, oxazolyl, isoxazolyl, triazolyl, thiadiazolyl, oxadiazolyl, tetrazolyl, thiatriazolyl, oxatriazolyl, pyridyl, pyrimidyl, imidazopyridyl, pyrazinyl, pyridazinyl, triazinyl, tetrazinyl, tetrazolol[1,5-b]pyridazinyl, purinyl, deazapurinyl, benzoxazolyl, benzofuryl, benzothiazolyl, benzothiadiazolyl, benzotriazolyl, benzoimidazolyl, indolyl, 1,3-thiazol-2-yl, 1,3,4-triazol-5-yl, 1,3-oxazol-2-yl, 1,3,4-oxadiazol-5-yl, 1,2,4-oxadiazol-5-yl, 1,3,4-thiadiazol-5-yl, 1H-tetrazol-5-yl, 1,2,3-triazol-5-yl, and pyrid-2-yl N-oxide. The term “heteroaryl” also includes groups in which a heteroaryl is fused to one or more cyclic (e.g., carbocyclyl, or heterocyclyl) rings, where the radical or point of attachment is on the heteroaryl ring. Nonlimiting examples include indolyl, indolizinyl, isoindolyl, benzothienyl, benzothiophenyl, methylenedioxyphenyl, benzofuranyl, dibenzofuranyl, indazolyl, benzimidazolyl, benzodioxazolyl, benzthiazolyl, quinolyl, isoquinolyl, cinnolinyl, phthalazinyl, quinazolinyl, quinoxalinyl, 4H-quinolizinyl, cardazolyl, acridinyl, phenazinyl, phenothiazinyl, phenoxazinyl, tetrahydroquinolinyl, tetrahydroisoquinolinyl and pyrido[2,3-b]-1,4-oxazin-3(4H)-one. A heteroaryl group may be mono-, bi- or tri-cyclic. In some embodiments, a heteroaryl group includes a heteroaryl ring fused to one or more (e.g., 1, 2 or 3) different cyclic groups (e.g., carbocyclic rings or heterocyclic rings), where the radical or point of attachment is on the heteroaryl ring, and in some embodiments wherein the point of attachment is a heteroatom contained in the heterocyclic ring. The structure of any heteroaryl group that is capable of having double bonds positioned differently is considered to embrace any and all such resonance structures. Heteroarylene” refers to a bivalent heteroaryl radical which may be optionally substituted.

### Treating Rhabdomyosarcoma Using PROTAC Compounds

In one aspect, the present invention provides a method of treating rhabdomyosarcoma in a subject in need thereof. The method includes administering a therapeutically effective amount of a PROTAC compound that results in the degradation of FOXO1 or a PAX3-FOXO1 fusion protein.

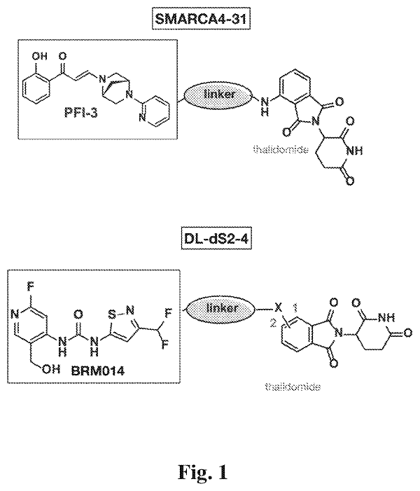

Proteolysis targeting chimeras (PROTACs) are hetero-bifunctional small molecules with three chemical elements: a ligand that recruits and binds to a target protein, a ligand that recruits and binds to E3 ubiquitin ligase, and a linker for conjugating these two ligands. PROTAC is a chemical knockdown strategy that degrades the target protein through the ubiquitin-proteosome system. Gao et al., ACS Med. Chem. Lett. 11, 237-240 (2020). FIG. 1 describes the modular constituents of two example PROTACs. Simultaneous binding of the target protein and the ligase by the PROTAC induces ubiquitylation of the target protein and its subsequent degradation by the ubiquitin-proteasome system, after which the PROTAC is recycled to degrade another target protein.

Ubiquitin-dependent proteolysis degrades intracellular proteins as part of normal cellular maintenance processes. In this process, proteins are targeted for degradation by the proteasome in a three-step process involving ubiquitin activating enzymes (E1), ubiquitin-conjugated enzymes (E2), and finally ubiquitin-protein ligases (E3), which coordinate the transfer of ubiquitin molecules to the target protein.

A PROTAC compound includes a ligand that binds to E3 ubiquitin ligase, also referred to herein as a “degron”. A ubiquitin pathway protein binding moiety is any suitable structure that recognizes and binds to a ubiquitin pathway protein. In general, a ubiquitin pathway protein is any entity or complex that is capable of catalyzing or causing to catalyze the transfer of a ubiquitin or ubiquitin-like modifying polypeptide, e.g., Nedd8, APG12 or ISG15/UCRP to another protein. In some embodiments, the ubiquitin pathway protein is a ubiquitin protein ligase or E3 protein. There are at least 100 distinct E3 proteins encoded by the human genome (See Winston, J., et al., Curr. Bio., 9:1180 82 (1999)). For example, one particular class of E3 proteins are Skp1-Cullin-F box (SCF) complexes, each complex containing Skp1, a member of Cullin family, e.g., the RING-12 protein Hrt1, also known as Roc1 or Rbx1, and an F box protein (See Deshaies, R. J., Annu. Rev. Cell Dev. Biol., 15:435 67 (1999)). These components are conserved from yeast to mammals.

A ubiquitin pathway protein binding moiety of is any suitable ligand to a ubiquitin pathway protein, e.g., ubiquitin protein ligase or E3 protein or homologs thereof. In another embodiment, a ubiquitin pathway protein binding moiety of the present invention is any ubiquitin pathway protein binding peptide, domain or region of a ligand to a ubiquitin pathway protein. In still another embodiment, a ubiquitin pathway protein binding moiety of the present invention recognizes and binds to a ubiquitin pathway protein in a regulated manner.

In some embodiments, the ligand that binds to E3 ubiquitin ligase is thalidomide or a thalidomide analog, which bind to cereblon (the protein product of the CRBN gene). Ito et al., Science, 327, 1345 (2010). Examples of thalidomide analogs include lenalidomide and pomalidomide. Thalidomide and its analogs act as degrader molecules (also known as molecular glues). Surka et al., Blood, 137, 661-677 (2021). These drugs co-opt the E3 ligase cereblon to target IKAROS family zinc finger 1 (IKZF1) and IKZF3 for degradation.

The PROTAC compound also includes a ligand that binds to a target protein, which can also be referred to as a targeting moiety. A targeting moiety is any structure that recognizes and binds to a target protein. The PROTAC compounds of the present invention can include targeting moieties that specifically bind to SMARCA4 and/or SMARCA2.

Targeting moieties are designed for specific binding, as a result of the affinity of complementary determining region of SMARCA4 and/or SMARCA2. A targeting moiety “specifically binds” when the targeting moiety preferentially binds a target structure, or subunit thereof, but binds to a substantially lesser degree or does not bind to a biological molecule that is not a target structure. In some embodiments, the targeting moiety specifically binds to the target analyte with a specific affinity of between 10⁻⁸ M and 10⁻¹¹ M. In some embodiments, the targeting moiety binds to the target structure with a specific affinity of greater than 10⁻⁷ M, 10⁻⁸ M, 10⁻⁹ M, 10⁻¹⁰ M, or 10⁻¹¹ M, between 10⁻⁸ M-10⁻¹¹ M, 10⁻⁹ M-10⁻¹⁰ M, and 10⁻¹⁰ M-10⁻¹¹ M. In a preferred aspect, specific activity is measured using a competitive binding assay as set forth in Ausubel FM, (1994). Current Protocols in Molecular Biology. Chichester: John Wiley and Sons (“Ausubel”), which is incorporated herein by reference.

In alveolar rhabdomyosarcoma, two pioneer factors, PAX3 and FOXO1, are fused in-frame in the recurrent translocation between chromosome arms 2p and 13q (Galili et al., Nat. Genet. 5, 230-235 (1993)). The resulting PAX3-FOXO1 fusion is an oncogenic driver that has been described as binding active regulatory elements alongside myogenic TFs (Gryder et al., Nat. Genet. 51, 1714-1722 (2019)), whereas its nucleosome targeting function in inactive or repressed chromatin domains remains unstudied. Neither retention of canonical pioneer activity nor the emergence of functions distinct from the wild-type PAX3 or FOXO1 monomers has been rigorously defined for PAX3-FOXO1 in fusion-positive rhabdomyosarcoma (FP-RMS). Given the relatively low mutational frequencies in FP-RMS, which can be approximated at 0.1 protein-coding mutations per Mb (Shern et al., Cancer Discov. 4, 216-231 (2014)), the inventors hypothesized that the pioneer function of PAX3-FOXO1, defined by targeting to nucleosomal motifs within inaccessible chromatin, might underlie its transforming potential in this tumor.

PAX3-FOXO1 is an oncogenic, chimeric transcription factor. Transcription factor, PAX3 plays an essential role in myogenesis. A recurrent chromosomal translocation results in the formation of a gene fusion, PAX3-FOXO1. The fusion consists of the first seven exons of PAX3 and the last two exons of FOXO. The fusion breakpoint typically occurs between exon 7 of the PAX3 coding sequence and exon 2 of FOXO1, although distinct breakpoints are observed in individual rhabdomyosarcoma patients and cell lines.

The amino acid sequence of PAX3-FPXP1 in human RH4 cells is provided below:

(SEQ ID NO: 1)
MTTLAGAVPRMMRPGPGQNYPRSGFPLEVSTPLGQGRVNQLGGVFINGR
PLPNHIRHKIVEMAHHGIRPCVISRQLRVSHGCVSKILCRYQETGSIRP
GAIGGSKPKQVTTPDVEKKIEEYKRENPGMFSWEIRDKLLKDAVCDRNT
VPSVSSISRILRSKFGKGEEEEADLERKEAEESEKKAKHSIDGILSERA
SAPQSDEGSDIDSEPDLPLKRKQRRSRTTFTAEQLEELERAFERTHYPD
IYTREELAQRAKLTEARVQVWFSNRRARWRKQAGANQLMAFNHLIPGGF
PPTAMPTLPTYQLSETSYQPTSIPQAVSDPSSTVHRPQPLPPSTVHQST
IPSNPDSSSAYCLPSTRHGFSSYTDSFVPPSGPSNPMNPTIGNGLSPQN
SIRHNLSLHSKFIRVQNEGTGKSSWWMLNPEGGKSGKSPRRRAASMDNN
SKFAKSRSRAAKKKASLQSGQEGAGDSPGSQFSKWPASPGSHSNDDFDN
WSTFRPRTSSNASTISGRLSPIMTEQDDLGEGDVHSMVYPPSAAKMAST
LPSLSEISNPENMENLLDNLNLLSSPTSLTVSTQSSPGTMMQQTPCYSF
APPNTSLNSPSPNYQKYTYGQSSMSPLPQMPIQTLQDNKSSYGGMSQYN
CAPGLLKELLTSDSPPHNDIMTPVDPGVAQPNSRVLGQNVMMGPNSVMS
TYGSQASHNKMMNPSSHTHPGHAQQTSAVNGRPLPHTVSTMPHTSGMNR
LTQVKTPVQVPLPHPMQMSALGGYSSVSSCNGYGRMGLLHQEKLPSDLD
GMFIERLDCDMESHRNDLMDGDTLDFNFDNVLPNQSFPHSVKTTTHSWV
SG.

In some embodiments, a protein other than PAX3 is included in a fusion protein together with FOXO1. This type of fusion protein is referred to herein as a DNA Binding Domain-FOXO1 fusion protein. FOXO1 fusions preserving the C-terminal amino acid sequence have been detected in stomach adenocarcinoma (WDFY2-FOXO1), lung adenocarcinoma (SMARCA4-FOXO1), and B-cell precursor acute lymphoblastic leukemia (MEIS1-FOXO1). Because these other fusion proteins can also be bound by PROTAC, they can also be used in methods of treating rhabdomyosarcoma and studying the chromatin-level effects of switching defective and sucrose nonfermenting inactivators, as described herein.

In some embodiments, the targeting moiety of the present invention should bind, or specifically bind, to SMARCA4 and/or SMARCA2. Proteolysis-targeting chimera (PROTAC) compounds have been developed for degrading the ATPase subunits SMARCA2 and SMARCA4 using a bromodomain ligand and recruitment of the E3 ubiquitin ligase VHL. High-resolution ternary complex crystal structures and biophysical investigation were used to optimize the compounds towards the compound ACB11, a potent and cooperative degrader of SMARCA2, SMARCA4 and PBRM1, ACB11 induced antiproliferative effects and cell death caused by SMARCA2 depletion on SMARCA4 mutant cancer cells and in acute myeloid leukemia cells dependent on SMARCA4 ATPase activity. Farnaby et al., Nat. Chem. Biol., 15(7), 672-680 (2019). Accordingly, in some embodiments, the PROTAC compound is SMARCA4-31 or DL-dS2-4, which are compounds that were identified by optimizing binding based on the compound ACB11.

A variety of PROTAC compounds including SMARCA2 and/or SMARCA4 binding regions have been described. See International Patent Publications WO 2020/0264172 and WO 2023/133260, the disclosures of which are incorporated herein by reference.

In some embodiments, the PROTAC comprises a compound according to Formula I or Formula II:

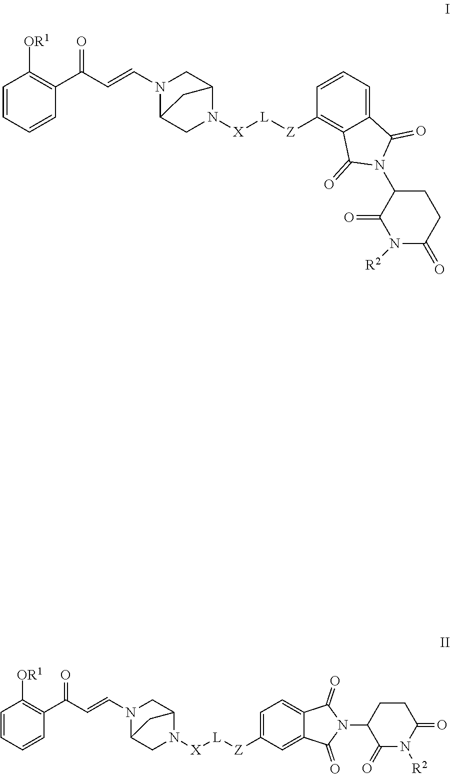

or a pharmaceutically acceptable salt thereof, wherein
X is a bond, aryl, or heteroaryl; L is an alkylene, alkenylene, or alkynylene chain comprising 1 to 35 carbon atoms, for example, 1 to 35 —CH₂— moieties, optionally wherein:
at least one, but no more than ten, —CH₂— moieties of L are independently replaced with a moiety selected from —C(═O)—, —C(═O)—NR₃—, —NR₃—C(═O)—, —C(═O)—O—, —O—C(═O)—, —NR₃—C(═O)—NR₃—, —O—C(═O)—NR₃—, —NR₃—C(═O)—O—, —O—, —S—, and —NR₃—, provided the number of —CH₂— moieties of L is larger than the collective number of —C(═O)—, —C(═O)—NR₃—, —NR₃—C(═O)—, —C(═O)—O—, —O—C(═O)—, —NR₃—C(═O)—NR₃—, —O—C(═O)—NR₃—, —NR₃—C(═O)—O—, —O—, —S—, and —NR₃— moieties of L, and
provided there is at least one —CH₂— between each —C(═O)—, —C(═O)—NR₃—, —NR₃—C(═O)—, —C(═O)—O—, —O—C(═O)—, —NR₃—C(═O)—NR₃—, —O—C(═O)—NR₃—, —NR₃—C(═O)—O—, —O—, —S—, and —NR₃— moiety of L;
Z-L is —CH₂-L, O—CH₂-L, or —NR₃—CH₂-L; and
R¹, R², and R³ are each independently selected from H and alkyl.

An example of a compound according to Formula I is SMARCA4-31, the structure of which is shown below:

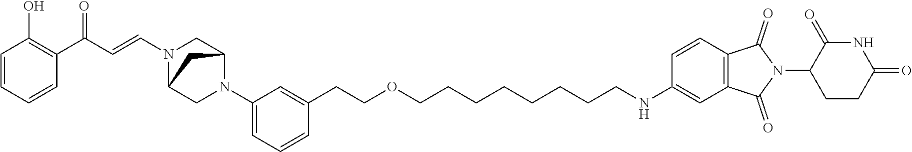 [SMILES: O=C1CCC(N2C(=O)c3ccc(NCCCCCCCCOCCc4cccc(N5C[C@H]6C[C@@H]5CN6/C=C/C(=O)c5ccccc5O)c4)cc3C2=O)C(=O)N1]

In some embodiments, the PROTAC compound comprises a structure according to formula III:

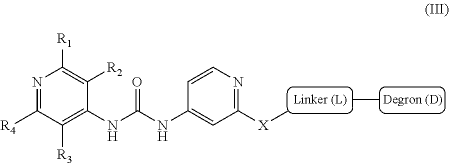

or a pharmaceutically acceptable salt or stereoisomer thereof, wherein
R₁ and R₄ are each independently H or halogen;
R₂ is H or NH₂;
R₃ is H, NH₂, NO₂, OH, CN, (C₁-C₆) alkyl, (C₁-C₆) hydroxyalkyl, (C₁-C₆) aminoalkyl, (C₃-C₆) carbocyclyl, 4- to 6-membered heterocyclyl. (C₁-C₆) alkyl-(C₃-C₆) carbocyclyl, or (C₁-C₆) alkyl-4- to 6-membered heterocyclyl, wherein said alkyl, hydroxyalkyl, aminoalkyl, carbocyclyl, or heterocyclyl is further optionally substituted by one or more, identical or different R₁ₐ groups, wherein each R₁ₐ is independently (C₁-C₆) alkyl, (C₁-C₆) alkoxy, (C₁-C₆) alkyl-(C₁-C₃) alkoxy, halogen, amino, hydroxyl, (C₁-C₆) haloalkyl, NH—(C₁-C₆) alkyl, N((C₁-C₆)alkyl)₂, (C₃-C₆) carbocyclyl, or 4- to 6-membered heterocyclyl;
X is C(O), C(O)NH, wherein

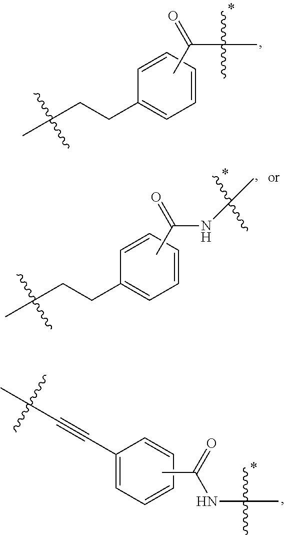

the squiggle is the point of attachment to the pyridine ring and the squiggle-asterisk [symbol]
is the point of attachment to the linker.

The PROTAC compounds described herein, including those of formula III, also include a degron (i.e., a ligand that binds to E3 ubiquitin ligase). Representative examples of degrons that bind to cereblon are represented by any one of structures (D1a) to (D1d):

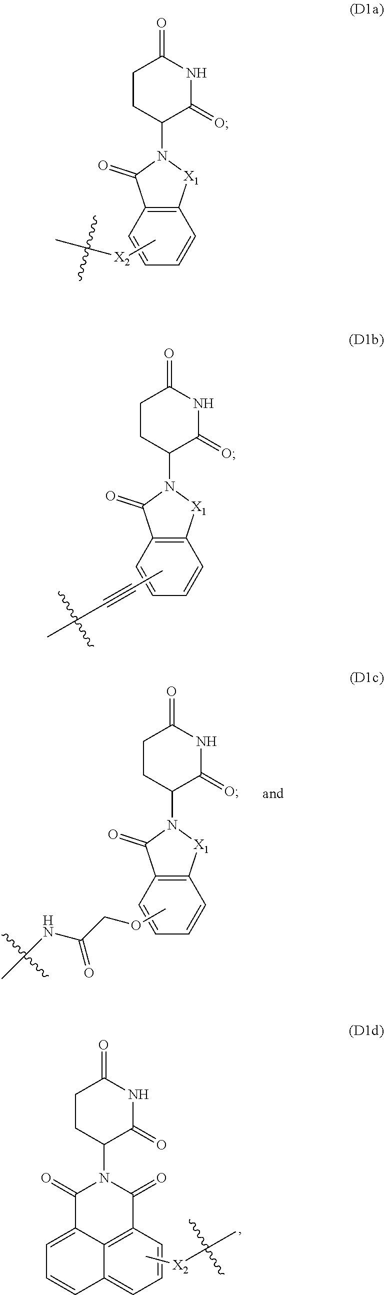

wherein X₁ is CEE or C(O) and X₂ is a bond, CH₂, NH, or O.

Yet other degrons that bind cereblon and which may be suitable for use in the present disclosure are disclosed in U.S. Pat. No. 9,770,512, and U.S. Patent Application Publication Nos. 2018/0015087, 2018/0009779, 2016/0243247, 2016/0235731, 2016/0235730, and 2016/0176916, and International Patent Publications WO 2017/197055, WO 2017/197051, WO 2017/197036, WO 2017/197056 and WO 2017/197046.

In some embodiments, the PROTAC compound is DL-dS2-4, the structure of which is shown below:

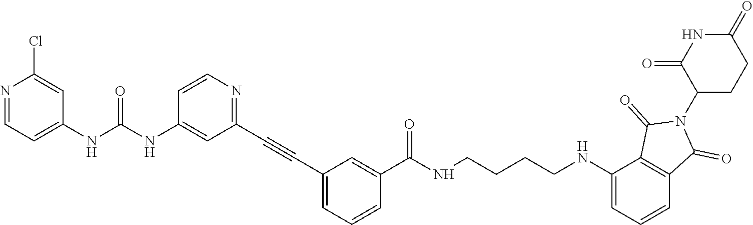 [SMILES: O=C1CCC(N2C(=O)c3cccc(NCCCCNC(=O)c4cccc(C#Cc5cc(NC(=O)Nc6ccnc(Cl)c6)ccn5)c4)c3C2=O)C(=O)N1]

The PROTAC compound also includes a linker which couples the ubiquitin pathway protein binding moiety to the targeting moiety. The structure of linker is not critical, provided it is substantially non-interfering with the activity of the PROTAC compound.

In some embodiments, the linker includes an alkylene chain (e.g., having 1-20 alkylene units). In some embodiments, the linker comprises an uninterrupted C₂ to C₁₅ alkylene chain. In other embodiments, the linker may include an alkylene chain, which may be interrupted by, and/or terminate (at either or both termini) at least one of —O—, —S—, —N(R′)—, —C═C—, —C(O)—, —C(O)O—, —OC(O)—, —OC(O)O—, —C(NOR′)—, —C(O)N(R′)—, —C(O)N(R′)C(O)—, —C(O)N(R′)C(O)N(R′)—, —N(R′)C(O)—, —N(R′)C(O)N(R′)—, —N(R′)C(O)O—, —OC(O)N(R′)—, —C(NR′)—, —N(R′)C(NR′)—, —C(NR′)N(R′)—, —N(R′)C(NR′)N(R′)—, —OB(Me)O—, —S(O)₂—, —OS(O)—, —S(O)O—, —S(O)—, —OS(O)₂—, —S(O)₂O—, —N(R′)S(O)₂—, —S(O)₂N(R′)—, —N(R′)S(O)—, —S(O)N(R′)—, —N(R′)S(O)₂N(R′)—, —N(R′)S(O)N(R′)—, C₃-C₁₂ carbocyclene, 3- to 12-membered heterocyclene, 5- to 12-membered heteroarylene or any combination thereof, wherein R₁ is H or C₁-C₆ alkyl, wherein the interrupting and the one or both terminating groups may be the same or different.

In some embodiments, the linker may include a C₂-C₁₅ alkylene chain terminating in NH-group wherein the nitrogen is also bound to the degron. In further embodiments, the linker comprises a C₆ carbocyclene, 6-membered heterocyclene, or phenyl group. In yet further embodiments, the linker may include a polyethylene glycol chain.

In some embodiments, the PROTAC compound further comprises a cell delivering moiety. A cell delivering moiety is any structure that facilitates the delivery of the composition or promotes transduction of the composition into cells. In one embodiment, a cell delivering moiety is derived from virus protein or peptide, e.g., a tat peptide. In another embodiment, a cell delivering moiety is a hydrophobic compound capable of penetrating cell membranes. Alternatively, a ubiquitin pathway protein binding moiety that is more susceptible for cell membrane penetration is used to enhance the cell membrane transduction of the composition. For example, n ubiquitin pathway protein binding peptide derived from the HIV Vpu protein.

The present invention provides a method of treating rhabdomyosarcoma in a subject in need thereof. Rhabdomyosarcoma is an aggressive and highly malignant form of cancer that develops from skeletal (striated) muscle cells that have failed to fully differentiate. It is generally considered to be a disease of childhood, as the vast majority of cases occur in those below the age of 18. Rhabdomyosarcoma can occur in any site on the body, but is primarily found in the head, neck, orbit, genitourinary tract, genitals, and extremities. Types of rhabdomyosarcoma include embryonal diabdomyosarcoma, alveolar rhabdomyosarcoma, and anaplastic rhabdomyosarcoma. In some embodiments, the rhabdomyosarcoma expresses high levels of the CRBN gene. Rhabdomyosarcoma is more likely to develop in patients having inherited gene mutations such as Li-Fraumeni syndrome. Neurofibromatosis type 1, DICER1 syndrome, Costello syndrome, Beckwith-Wiedemann syndrome, Noonan syndrome, and cardio-facio cutaneous syndrome.

In some embodiments, the subject is a human who has been diagnosed with rhabdomyosarcoma. Rhabdomyosarcoma can be difficult to diagnose due to its similarities to other cancers and varying levels of differentiation, but is typically diagnosed based on the use of imaging tests such as CT scans, MRI, and PET scans, followed by a biopsy. It is loosely classified as one of the “small, round, blue-cell cancer of childhood” due to its appearance on an H&E stain. However, the defining diagnostic trait for rhabdomyosarcoma is confirmation of malignant skeletal muscle differentiation with myogenesis under light microscopy. Magnetic resonance imaging (MRI), ultrasonography, and a bone scan can be used to determine the extent of local invasion and metastasis.

The method of treatment comprises administering a therapeutically effective amount of a PROTAC compound that results in the degradation of FOXO1 or a PAX3-FOXO1 fusion protein. In some embodiments, a plurality of different PROTAC compounds that result in the degradation of FOXO1 or a PAX3-FOXO1 fusion protein are administered. In some embodiments, the PROTAC compound is administered in a pharmaceutically acceptable carrier.

The present invention includes the use therapeutic targets that contribute to indirect interference with PAX3-FOXO1 activity in rhabdomyosarcoma at the different molecular levels. Examples of therapeutic targets include upstream modifiers and activators, epigenetic and transcriptional co-regulators, and downstream effector targets. In some embodiments, the genomic region is at least a portion of a gene. In some embodiments, the interference is the degradation of the ATPase subunits SMARCA2 and SMARCA4. The present invention includes a variety of methods of modulating the expression of a genomic region of a cancer cell. For examples of such methods, see Wachtel M. and Schafer B W, Seminars in Cancer Biology, 50, 115 (2018) and Nguyen T. and Barr F., Molecules, 23(11), 2798 (2018). These methods can be used alone, or in combination with known methods of treating rhabdomyosarcoma such as chemotherapy. The expression of the genomic region is modulated (i.e., increased or decreased) by administering an effective amount of a nucleic acid. The nucleic acid may be included in a delivery system enabling efficient intracellular introduction. The delivery system may be preferably a vector, and both viral vector and non-viral vector may be used. The viral vector may include lentivirus, retrovirus, adenovirus, herpes virus and avipox virus vector, and the like may be used, but is not limited thereto.

The amino acid sequence of the protein expressed by the human SMARCA2 gene is as follows:

(SEQ ID NO: 2)
XEEEKIFGRGSRQRRDVDYSDALTEKQWLRAIEDGNLEEMEEEVRLKKR
KRRRNVDKDPAKEDVEKAKKRRGRPPAEKLSPNPPKLTKQMNAIIDTVI
NYKDSSGRQLSEVFIQLPSRKELPEYYELIRKPVDFKKIKERIRNHKYR
SLGDLEKDVMLLCHNAQTFNLEGSQIYEDSIVLQSVEKSARQKIAKEEE
SEDESNEEEEEEDEEESESEAKSVKVKIKLNKKDDKGRDKGKGKKRPNR
GKAKPVVSDFDSDEEQDERVSVADWD

A number of sequences for the protein expressed by the human SMARCA4 gene are known. One example human sequence is as follows:

(SEQ ID NO: 3)
MSTPDPPLGGTPRPGPSPGPGPSPGAMLGPSPGPSPGSAHSMMGPSPGP
PSAGHPIPTQGPGGYPQDNMHQMHKPMESMHEKGMSDDPRYNQMKGMGM
RSGGHAGMGPPPSPMDQHSQGYPSPLGGSEHASSPVPASGPSSGPQMSS
GPGGAPLDGA

In some embodiments of the method of treatment, the expression of the genomic region is decreased. Genetic methods such as the use of siRNA, ribozymes, or antisense RNA could also be used to suppress expression of a genomic region. For example, the expression can be decreased by administering an effective amount of a siRNA to the subject. siRNA is a duplex RNA which specifically cleaves target molecules to induce RNA interference (RNAi). Preferably, the siRNA of the present invention has a nucleotide sequence composed of a sense RNA strand homologous entirely or partially to a gene expressing a mutant NRF2 pathway protein nucleic acid sequence and an antisense RNA strand complementary thereto, which hybridizes with its target sequence within cells.

In some embodiments, treatment of rhabdomyosarcoma includes the use of methods in addition to the use administering a therapeutically effective amount of a PROTAC compound. Additional methods of treatment include surgery, chemotherapy, radiation, and immunotherapy. Chemotherapy has been shown to be the most effective method for treating rhabdomyosarcoma. The two main chemotherapeutic methods for the treatment of rhabdomyosarcoma are the VAC regimen, consisting of vincristine, actinomycin D. and cyclophosphamide, and the IVA regimen, consisting of ifosfamide, vincristine, and actinomycin D.

### Understanding ATP-Dependent Chromatin Remodeling

As PROTAC compounds such as SMARCA4-31 and DL-dS2-4 target the ATPase subunits of the SWI/SNF chromatin remodeling complex (e.g., SMARCA4 (BRG1) and SMARCA2 (BRM)) these compounds can be used for studying the chromatin-level effects of SWI/SNF inactivation by PROTAC-mediated ATPase degradation. Upon ATPase degradation by PROTACs. SWI/SNF complexes are disassembled and rendered non-functional, and their central role in ATP-dependent chromatin remodeling is eliminated.

Accordingly, another aspect of the invention provides a method of studying the chromatin-level effects of switching defective and sucrose nonfermenting (SWI/SNF) inactivators, comprising degrading ATPase by contacting it with an effective amount of a proteolysis-targeting chimera (PROTAC) compound, and measuring the biological effect of the loss of SWI/SNF complexes. A biological effect can be, for example, a biological change in a subject, such as a test animal or animal model, or a biochemical effect in an in vitro test.

The PROTAC compound can be any of the PROTAC compounds described herein, including any of the compounds of Formulas I, II, or Ill. In some embodiments, the PROTAC compound includes a targeting ligand that specifically binds to SMARCA4 and/or SMARCA2. In some embodiments, the PROTAC compound is SMARCA4-31 or DL-dS2-4.

The method of studying the chromatin-level effects of switching defective and sucrose nonfermenting (SWI/SNF) inactivators can be carried out in vivo or in vitro. In some embodiments, the method is conducted in vitro. In vitro analysis includes ex Nito analysis in cultured cells.

In some embodiments, measuring the biological effects comprises characterizing genome-wide nucleosome positioning. For example, one could directly measure the impacts of this loss by characterizing genome-wide nucleosome positioning using MNase-seq or genome-wide chromatin accessibility using ATAC-seq.

In some embodiments, measuring the biological effects comprises measuring genome-wide residence of chromatin-bound proteins using ChIP-seq or changes to gene expression and splicing using RNA-seq. For example, one could measure indirect results of altered ATP-dependent chromatin remodeling by measuring genome-wide residence of chromatin bound proteins as well as post-translational modifications to histone proteins that comprise chromatin using ChIP-seq or changes to gene expression and splicing using RNA-seq.

In further embodiments, measuring the biological effects comprises quantification of three-dimensional chromatin conformation changes by Hi-C, while in yet further embodiments measuring the effects comprises quantification of cell morphological changes by immunofluorescence microscopy.

### Formulation and Administration

The present invention provides methods comprising administration a therapeutically effective amount of a PROTAC compound in a pharmaceutical composition. Examples of pharmaceutical compositions include those for oral, intravenous, intramuscular, subcutaneous, transdermal, or intraperitoneal administration, or any other route known to those skilled in the art, and generally involves providing the PROTAC compound formulated together with a pharmaceutically acceptable carrier. The dosage form should be effective for therapeutic use and may be, for example, a solid preparation (e.g., a tablet(s)), a liquid preparation (e.g., oral suspension), or an injection (e.g., an intravenous injection).

The dose, dosing interval, and administration method of a therapeutically effective amount of a PROTAC compound may be selected. The amount of PROTAC compound that is administered and the dosage regimen for treating a disease condition with the compounds and/or compositions of this invention depends on a variety of factors, including the age, weight, sex, and medical condition of the subject, the severity of the disease, the route and frequency of administration, and the particular compound employed, the location of the unwanted proliferating cells, as well as the pharmacokinetic properties of the individual treated, and thus may vary widely. The dose may be, for example, from 0.01 to 200 mg/kg body weight per administration, or from 0.05 to 10 mg/kg body weight. The dosing interval, for example, may be 1 or 2 doses for 1 to 28 days, for 1 to 4 weeks, or from 1 to 4 months. More specifically, 20 to 80 mg/day intravenous injection may be included. Also included is 20 to 80 mg/day intravenous injection twice a week. At the time of the therapy, the PROTAC compound dose may include 10 to 40 mg or 10 to 20 mg/day intravenous injection. Also included is 10 to 40 mg/day or 10 to 20 mg/day intravenous injection twice a week. The “range of 10 to 80 mg” may mean, for instance, 10, 20, 30, 40, 50, 60, 70, or 80 mg or a number between any two thereof.

When preparing a PROTAC compound for oral administration, the pharmaceutical composition may be in the form of, for example, a tablet, capsule, suspension or liquid. The pharmaceutical composition is preferably made in the form of a dosage unit containing a particular amount of the active ingredient. Examples of such dosage units are capsules, tablets, powders, granules or a suspension, with conventional additives such as lactose, mannitol, corn starch or potato starch; with binders such as crystalline cellulose, cellulose derivatives, acacia, corn starch or gelatins; with disintegrators such as corn starch, potato starch or sodium carboxymethyl-cellulose; and with lubricants such as talc or magnesium stearate. The active ingredient may also be administered by injection as a composition wherein, for example, saline, dextrose or water may be used as a suitable carrier.

For intravenous, intramuscular, subcutaneous, or intraperitoneal administration, the compound may be combined with a sterile aqueous solution which is preferably isotonic with the blood of the recipient. Such formulations may be prepared by dissolving solid active ingredient in water containing physiologically compatible substances such as sodium chloride, glycine, and the like, and having a buffered pH compatible with physiological conditions to produce an aqueous solution, and rendering said solution sterile. The formulations may be present in unit or multi-dose containers such as sealed ampoules or vials.

Formulations suitable for parenteral administration conveniently comprise a sterile aqueous preparation of the active compound which is preferably made isotonic. Preparations for injections may also be formulated by suspending or emulsifying the compounds in non-aqueous solvent, such as vegetable oil, synthetic aliphatic acid glycerides, esters of higher aliphatic acids or propylene glycol.

The PROTAC compound can also be provided as a pharmaceutically acceptable salt. The phrase “pharmaceutically acceptable salts” connotes salts commonly used to form alkali metal salts and to form addition salts of free acids or free bases. The nature of the salt is not critical, provided that it is pharmaceutically acceptable. Suitable pharmaceutically acceptable acid addition salts of the compounds may be prepared from an inorganic acid or from an organic acid.

Examples have been included to more clearly describe a particular embodiment of the invention and its associated cost and operational advantages. However, there are a wide variety of other embodiments within the scope of the present invention, which should not be limited to the particular example provided herein.

### EXAMPLES

### Example 1: Targeted Degradation of BAF-Associated Transcription Factors in Rhabdomyosarcoma

The inventors investigated the effect of a number of PROTAC inhibitors on fusion-positive and negative RMS cell models. The experiment was designed as follows. Fusion-positive RMS (RH4 and RH30) and fusion-negative RMS (RD and CTR) cells were treated with DMSO or 1 μM dBRD9, DL-ds2-4, or SMARCA4-31 for 24 hours. Soluble nuclear extracts were prepared to assess target degradation as well as abundance of additional SWI/SNF complex subunits and the transcription factor FOXO1 (wild-type and PAX3-FOXO1 fusion).

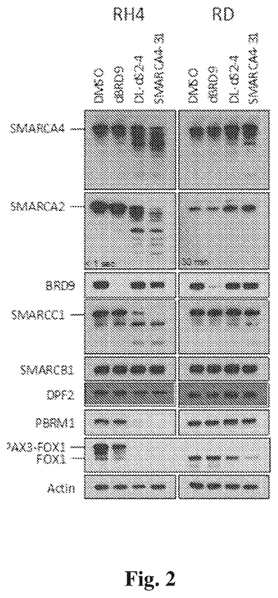

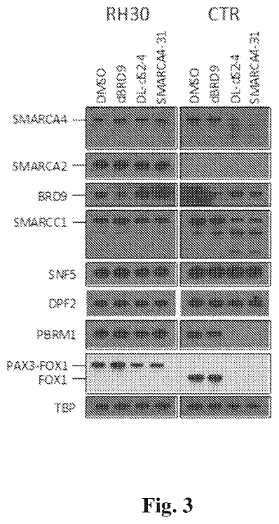

The results showed that RH4 and CTR cells showed significant on-target activity in these conditions, exhibiting degradation of BRD9, SMARCA2, and SMARCA4. RD and RH30 showed BRD9 degradation of BRD9, but little evidence of ATPase degradation. ATPase degraders have potent cytotoxicity. See FIGS. 2 and 3.

In addition to on-target activity, LD-d2-4 and SMARCA4-31 treatment degraded the core WI/SNF subunit SMARCC1 while second core subunit of the BAF and PBAF complexes, SMARCB1, remained intact. Additionally, DL-d52-4 and SMARCA4-31 resulted in specific degradation of the dedicated PBAF subunit, PBRM1, while the BAF subunit DPF2 and GBAF subunit BRD9 remained intact. In RH4 cells, which express the PAX3-FOXO1 fusion transcription factor, DL-dS2-4 and SMARCA4-31 treatment resulted in loss of the PAX3-FOXO1 fusion. In CTR cells, which only express the wild-type FOXO1, DL-dS2-4 and SMARCA4-31 treatment resulted in FOXO1 loss.

The inventors interpret these results as follows. The high sensitivity of RH4 and CTR cells to ATPase degradation may be the result of broad effects these compounds have on major SWI/SNF subunits and sub-complexes as well as their effect on critical transcription factor networks (i.e., PAX3-FOXO1) and suggests therapeutic implications. PBRM1 degradation phenotype is consistent with Farnaby et al., Nat. Chem Bio. 15, 672-680 (2019), though SMARCC1 degradation is a novel phenotype. The inventors do not yet understand the differential sensitivity of RMS cells to these.

The inventors also investigated whether the effect of the compounds was reversible. Fusion-positive RMS RH4 cells were treated for 24 hours with DMSO including 1 mM dBRD9, DL-ds2-4, or SMARCA4-31. After the treatment period cells were collected or washed extensively to remove the compound and incubated in untreated growth media for an additional 24-hour period. Soluble nuclear extracts were prepared to assess target degradation and recovery.

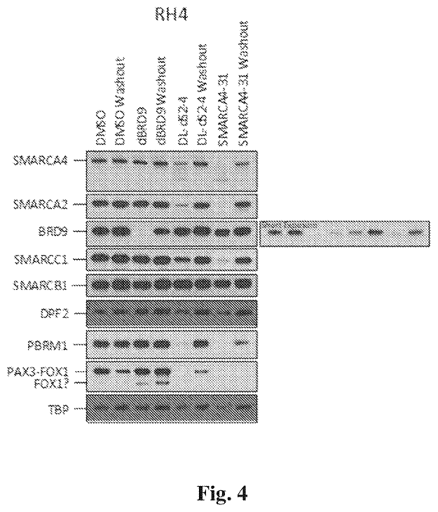

On target activity of the compounds in RH4 cells over 24 hours is shown in FIG. 4 and is in agreement with the results of FIGS. 2 and 3. In most cases, proteins lost as a result of degrader treatment recover nearly to pre-treatment levels. More complete degradation of SMARCA4, PBRM1, and PAX3-FOXO1 after SMARCA4-31 treatment was associated with less complete recovery over the 24-hour washout period, or no recovery at all in the case of PAX3-FOXO1. These results demonstrate that the compounds are reversible, which is an important trait for their use as a research tool for understanding complex reformation and redeployment to the genome.

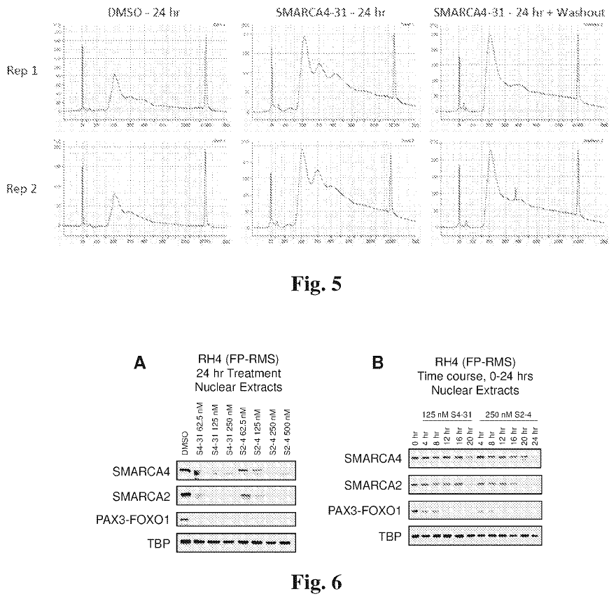

Finally, the inventors determined if the reversible disruption of the SWU/SNF complex results in altered chromatin accessibility. FP-RMS RH4 cells were treated for 24 hours with DMSO or SMARCA4-31. A third group was treated with SMARCA4-31 for 24 hours before washout, and a 24-hour recovery period. 50.000 cells were used. Tn5 transposition followed by Illumina library amplification (15 cycles). Libraries were run on Bioanalyzer to assess fragment length distribution. The results (See FIG. 5) show reproducible fragment length signatures distinguished treatment conditions, suggesting alterations to the accessibility landscape in RH4 cells.

### Example 2: Additional Evidence of Destabilization of BAF Associated Transcription Factors by PROTAC Treatment

Our initial western blot data demonstrated concurrent loss of ATPases SMARCA4/SMARCA2 and the transcription factors PAX3-FOXO1 and FOXO1 following a 24-hour treatment of rhabdomyosarcoma cells with 1 μM SMARCA4-31 or DL-dS2-4. Our initial data also showed by liquid chromatography mass spectrometry (LC/MS) that additional SWI/SNF (BAF)-associated transcription factors are depleted upon 24-hour treatment of rhabdomyosarcoma cells with 1 μM SMARCA4-31. In addition to PAX3-FOXO1, depleted core-regulatory transcription factors included MYOD1, an essential factor in rhabdomyosarcoma cell growth.

We have since performed additional studies demonstrating full PAX3-FOXO1 loss at lower PROTAC doses and over shorter treatment periods than is required for complete ATPase loss.

In FIG. 6A, RH4 cells were treated for 24 hours with the indicated dose of SMARCA4-31 (labeled S4-31) or DL-dS2-4 (labeled S2-4). The data show that while 125 nM of SMARCA4-31 or 250 nM DL-dS2-4 is required for maximal ATPase depletion, PAX3-FOXO1 is fully depleted at a PROTAC concentration of 62.5 nM.

In FIG. 6B, RH4 cells were treated with 125 nM SMARCA4-31 (labeled S4-31) or 250 nM DL-dS2-4 (labeled S2-4) for the indicated time. The data show that while ATPase depletion is not observed until approximately 20 hours of PROTAC exposure, complete PAX3-FOXO1 loss is observed within approximately 12 hours of PROTAC exposure.

Taken together, these data suggest that SMARCA4-31 and DL-dS2-4, in addition to targeting ATPases for degradation at sufficient doses and exposure periods, also result in the relatively rapid loss of PAX3-FOXO1 in rhabdomyosarcoma cells at low concentrations.

### Example 3: Additional Evidence of Rhabdomyosarcoma Cell Sensitivity to PROTAC Treatment

The concurrent loss of SMARCA4 and SMARCA2, along with essential BAP-associated transcription factors including PAX3-FOXO1 and MYOD1 upon PROTAC exposure is expected to result in a marked decrease in rhabdomyosarcoma cell viability. Below, we have provided initial data demonstrating sub-micromolar efficacy of SMARCA4-31 and DL-dS2-4 in inhibiting growth of two rhabdomyosarcoma cell lines, RH4 and SMS-CTR.

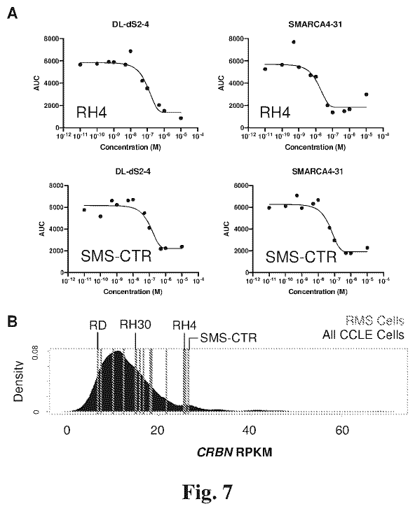

In FIG. 7A, RH4 (a PAX3-FOXO1 fusion-positive rhabdomyosarcoma cell line) and SMS-CTR (a PAX3-FOXO1 fusion-negative rhabdomyosarcoma cell line) cells were treated with increasing concentrations of SMARCA4-31 or DL-dS2-4 for a 96-hour period, and cell proliferation was monitored on an Incucyte® system. The Area Under the Growth Curve (AUC) at each PROTAC concentration was used to generate a dose-response curve demonstrating rhabdomyosarcoma cell growth inhibition at sub-micromolar PROTAC concentrations. We explored why RH4 and SMS-CTR cells display relatively high sensitivity to PROTAC treatment. The CRBN gene expresses the protein cereblon, a component of an E3 ubiquitin ligase complex. By analyzing CRBN gene expression using RNA-seq from the Cancer Cell Line Encyclopedia (CCLE) database, we show in FIG. 7B, that RH4 and SMS-CTR cells express high levels of CRBN compared to other rhabdomyosarcoma (RMS) cell lines. As thalidomide-based PROTACs function through CRBN-dependent recruitment of target proteins to the CRL4 ubiquitination apparatus, we suggest the sensitivity of RH4 and SMS-CTR cells to PROTACs is due to their high expression of CRBN.

The complete disclosure of all patents, patent applications, and publications, and electronically available material cited herein are incorporated by reference. The foregoing detailed description and examples have been given for clarity of understanding only. No unnecessary limitations are to be understood there from. The invention is not limited to the exact details shown and described, for variations obvious to one skilled in the art will be included within the invention defined by the claims.

What is claimed is:

1. A method of treating rhabdomyosarcoma in a subject in need thereof, comprising administering a therapeutically effective amount of a proteolysis-targeting chimera (PROTAC) compound that results in the degradation of FOXO1 or a PAX3-FOXO1 fusion protein.

2. The method of claim 1, wherein the PROTAC compound specifically binds to SMARCA4 and/or SMARCA2.

3. The method of claim 1, wherein the PROTAC comprises a compound according to Formula I or Formula II: 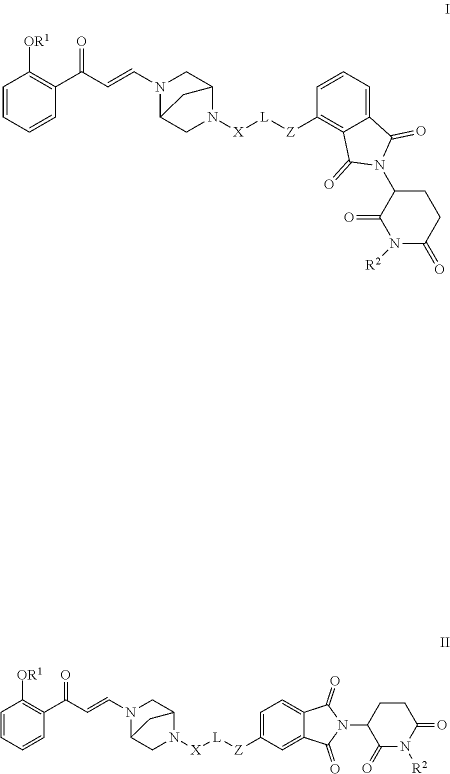
or a pharmaceutically acceptable salt thereof, wherein
X is a bond, aryl, or heteroaryl; L is an alkylene, alkenylene, or alkynylene chain comprising 1 to 35 carbon atoms, for example, 1 to 35 —CH₂— moieties, optionally wherein:
at least one, but no more than ten, —CH₂— moieties of L are independently replaced with a moiety selected from —C(═O)—, —C(═O)—NR₃—, —NR₃—C(═O)—, —C(═O)—O—, —O—C(═O)—, —NR₃—C(═O)—NR₃—, —O—C(═O)—NR₃—, —NR₃—C(═O)—O—, —O—, —S—, and —NR₃—, provided the number of —CH₂— moieties of L is larger than the collective number of —C(═O)—, —C(═O)—NR₃—, —NR₃—C(═O)—, —C(═O)—O—, —O—C(═O)—, —NR₃—C(═O)—NR₃—, —O—C(═O)—NR₃—, —NR₃—C(═O)—O—, —O—, —S—, and —NR₃— moieties of L, and
provided there is at least one —CH₂— between each —C(═O)—, —C(═O)—NR₃—, —NR₃—C(═O)—,
—C(═O)—O—, —O—C(═O)—, —NR₃—C(═O)—NR₃—, —O—C(═O)—NR₃—, —NR₃—C(═O)—O—, —O—, —S—, and —NR₃— moiety of L;
Z-L is —CH₂-L, O—CH₂-L, or —NR₃—CH₂-L; and
R¹, R², and R³ are each independently selected from H and alkyl.

4. The method of claim 3, wherein the PROTAC compound is SMARCA4-31

5. The method of claim 1, wherein the compound has a structure according to formula III: 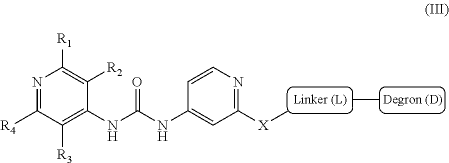
or a pharmaceutically acceptable salt or stereoisomer thereof, wherein
R₁ and R₄ are each independently H or halogen;
R₂ is H or NH₂;
R₃ is H, NH₂, NO₂, OH, CN, (C₁-C₆) alkyl, (C₁-C₆) hydroxyalkyl, (C₁-C₆) aminoalkyl, (C₃-C₆) carbocyclyl, 4- to 6-membered heterocyclyl, (C₁-C₆) alkyl-(C₃-C₆) carbocyclyl, or (C₁-C₆) alkyl-4- to 6-membered heterocyclyl, wherein said alkyl, hydroxyalkyl, aminoalkyl, carbocyclyl, or heterocyclyl is further optionally substituted by one or more, identical or different Ra groups, wherein each R₁ₐ is independently (C₁-C₆) alkyl, (C₁-C₆) alkoxy, (C₁-C₆) alkyl-(C₁-C₃) alkoxy, halogen, amino, hydroxyl, (C₁-C₆) haloalkyl, NH—(C₁-C₆) alkyl, N((C₁-C₆)alkyl)₂, (C₃-C₆) carbocyclyl, or 4- to 6-membered heterocyclyl;
X is C(O), C(O)NH, wherein 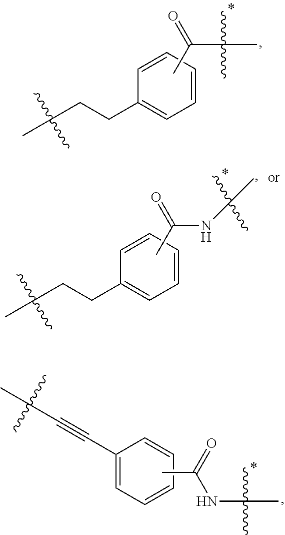
the squiggle is the point of attachment to the pyridine ring and the squiggle-asterisk [symbol]
is the point of attachment to the linker.

6. The method of claim 5, wherein the degron is a compound selected from D1a-D1d: 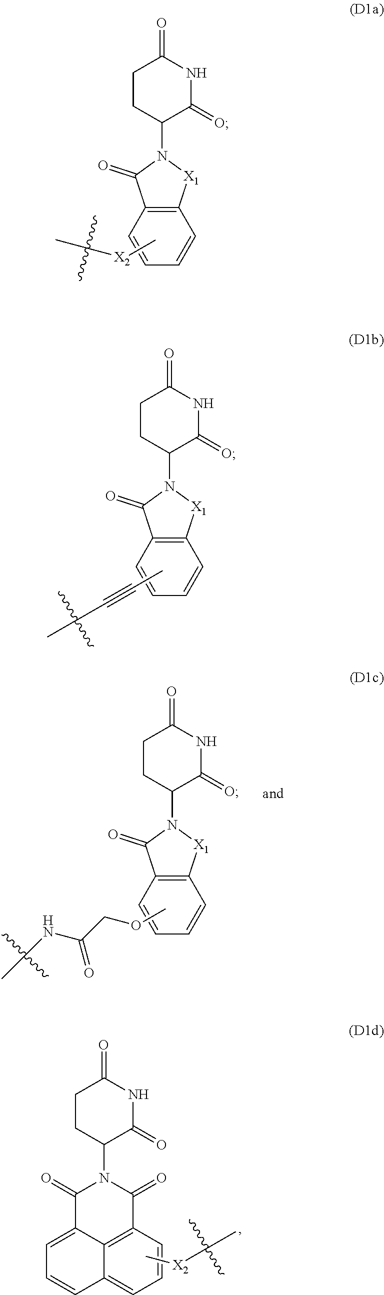
wherein X₁ is CEE or C(O) and X₂ is a bond, CH₂, NH, or O.

7. The method of claim 5, wherein the PROTAC compound is DL-dS2-4.

8. The method of claim 1, wherein the PROTAC compound comprises thalidomide or a thalidomide analog.

9. The method of claim 1, wherein the subject is a human who has been diagnosed with rhabdomyosarcoma.

10. The method of claim 1, wherein the PROTAC compound is administered with a pharmaceutically acceptable carrier.

11. A method of studying the chromatin-level effects of switching defective and sucrose nonfermenting (SWI/SNF) inactivators, comprising degrading ATPase by contacting it with an effective amount of a proteolysis-targeting chimera (PROTAC) compound, and measuring the biological effect of the loss of SWI/SNF complexes.

12. The method of claim 11, wherein the PROTAC compound is SMARCA4-31 or DL-dS2-4.

13. The method of claim 11, wherein the method is conducted in vitro.

14. The method of claim 11, wherein measuring the biological effects comprises characterizing genome-wide nucleosome positioning.

15. The method of claim 11, wherein measuring the biological effects comprises measuring genome-wide residence of chromatin-bound proteins using ChIP-seq or changes to gene expression and splicing using RNA-seq.

16. The method of claim 11, wherein measuring the biological effects comprises quantification of three-dimensional chromatin conformation changes by Hi-C.

17. The method of claim 11, wherein measuring the biological effects comprises quantification of cell morphological changes by immunofluorescence microscopy.
